(19) United States
(12) Patent Application Publication
(10) Pub. No.: US 2026/0170250 A1
(43) Pub. Date: Jun. 18, 2026

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Motoki TANIGUCHI, Tokyo (JP)

(73) Assignee: FUJIFILM Corporation, Tokyo (JP)

(21) Appl. No.: 19/411,346

(22) Filed: Dec. 7, 2025

(30) Foreign Application Priority Data
Dec. 13, 2024 (JP) 2024-219369

(51) Int. Cl. G06F 40/289; G06F 40/247; G16H 30/00

(52) U.S. Cl. CPC G06F 40/289; G06F 40/247; G16H 30/00

(57) ABSTRACT

A processor acquires a sentence, derives structured information by structuring the sentence, acquires classification information regarding a term included in the sentence, and normalizes the structured information based on the classification information.

### CROSS REFERENCE TO RELATED APPLICATIONS

The present application claims priority from Japanese Patent Application No. 2024-219369, filed on Dec. 13, 2024, the entire disclosure of which is incorporated herein by reference.

### BACKGROUND

### Technical Field

The present disclosure relates to an information processing apparatus, an information processing method, and an information processing program.

### Related Art

Sentences created in medical settings are text freely described by medical workers such as doctors, and as they are, the sentences are unstructured data that is difficult to use for secondary purposes such as statistical analysis or content analysis. In an interpretation report, which is a type of medical document, a result obtained by a doctor after observing images captured by a medical apparatus such as a computed tomography (CT) apparatus or a magnetic resonance imaging (MRI) apparatus, and understanding properties, such as the location, size, shape, or internal structure of each disease, is described as a comment on findings. In order to acquire this information described in the interpretation report, various methods have been proposed to structure the sentence by extracting terms of classes (attributes) such as anatomy, lesion, size of the lesion, properties, and disease name from the comment on findings included in the interpretation report.

For example, JP2023-114341A proposes a method of classifying attributes of information described in a sentence into certain units of the sentence, analyzing the sentence for each same classification based on a result of the classification, and outputting a result of the analysis.

On the other hand, the way in which a sentence is expressed in the interpretation report varies depending on the medical worker. Therefore, synonyms of the terms extracted by structuring are searched for in a synonym dictionary prepared in advance, and normalization is performed to convert the searched synonyms into representative notations (or dictionary codes). Normalization makes it possible to absorb variations in the notation of terms in a sentence and standardize the notation of terms, thereby facilitating the search and analysis of interpretation reports.

Depending on the terms used in the sentence, a plurality of terms may be searched for as candidates for normalization. In such a case, the terms cannot be normalized.

### SUMMARY OF THE INVENTION

The present disclosure has been made in consideration of the above circumstances, and an object of the present disclosure is to enable accurate normalization of structured information such as terms derived by structuring a sentence.

An information processing apparatus according to an aspect of the present disclosure comprises a processor,
in which the processor is configured to:
acquire a sentence;
derive structured information by structuring the sentence;
acquire classification information regarding a term included in the sentence; and
normalize the structured information based on the classification information.

In the information processing apparatus according to the aspect of the present disclosure, the classification information may include at least one of a synonym of the structured information or information on a field related to the sentence.

In the information processing apparatus according to the aspect of the present disclosure, the sentence may be a medical sentence, and the information on the field related to the sentence may include at least one of an organ, a part, a modality that has acquired a medical image for which the sentence is created, or a medical department that uses the medical image.

In the information processing apparatus according to the aspect of the present disclosure, the processor may be configured to normalize the structured information with reference to a synonym dictionary that has been classified according to the classification information.

In the information processing apparatus according to the aspect of the present disclosure, the processor may be configured to, in a case in which the structured information is not able to be normalized with reference to the synonym dictionary, complement the structured information using a rule corresponding to a field related to the sentence, and normalize the complemented structured information.

In the information processing apparatus according to the aspect of the present disclosure, the processor may be configured to output a basis for normalizing the structured information.

In the information processing apparatus according to the aspect of the present disclosure, the processor may be configured to display the normalized structured information.

In the information processing apparatus according to the aspect of the present disclosure, the processor may be configured to, in a case in which the structured information is able to be normalized without using the classification information, normalize the structured information without using the classification information, and display the structured information normalized without using the classification information and the structured information normalized using the classification information in a distinguishable manner.

An information processing method according to another aspect of the present disclosure comprises:
acquiring, by a computer, a sentence;
deriving, by the computer, structured information by structuring the sentence;
acquiring, by the computer, classification information regarding a term included in the sentence; and
normalizing, by the computer, the structured information based on the classification information.

An information processing program according to another aspect of the present disclosure causes a computer to execute:
a step of acquiring a sentence;
a step of deriving structured information by structuring the sentence;
a step of acquiring classification information regarding a term included in the sentence; and
a step of normalizing the structured information based on the classification information.

The technology of the present disclosure may be provided as a program product.

According to the aspects of the present disclosure, it is possible to accurately normalize structured information derived by structuring a sentence.

### BRIEF DESCRIPTION OF THE DRAWINGS

FIG. 1 is a diagram showing a schematic configuration of an information processing system to which an information processing apparatus according to the present embodiment is applied.

FIG. 2 is a block diagram showing a hardware configuration of the information processing apparatus according to the present embodiment.

FIG. 3 is a block diagram showing a functional configuration of the information processing apparatus according to the present embodiment.

FIG. 4 is a diagram for describing structuring.

FIG. 5 is a diagram showing an example of a synonym dictionary.

FIG. 6 is a diagram showing a display screen of a derivation result of a representative notation in a first embodiment.

FIG. 7 is a flowchart showing processing performed in the first embodiment.

FIG. 8 is a diagram showing an example of a template used in a second embodiment.

FIG. 9 is a diagram showing a display screen of a derivation result of a representative notation in the second embodiment.

FIG. 10 is a flowchart showing processing performed in the second embodiment.

FIG. 11 is a diagram showing another example of a display screen of a derivation result of a representative notation.

### DETAILED DESCRIPTION

An example of embodiments of the disclosed technology will be described below with reference to the drawings. Note that the same or equivalent components and parts in each drawing are given the same reference numerals, and duplicated descriptions will be omitted. Further, dimensional ratios in the drawings are exaggerated for convenience of description, and may be different from the actual ratios.

First, an information processing system 1 to which an information processing apparatus 20 according to a first embodiment of the present disclosure is applied will be described with reference to FIG. 1. FIG. 1 is a diagram showing a schematic configuration of the information processing system 1. The information processing system 1 is a system for analyzing an interpretation report created by interpreting medical images acquired at a medical institution or the like.

Specifically, the information processing system 1 includes a medical institution system 10 constructed in a medical institution such as a hospital, and the information processing apparatus 20. The medical institution system 10 and the information processing apparatus 20 are connected to each other in a communicable state via a wired or wireless network 9. The network 9 is, for example, a network such as a local area network (LAN) and a wide area network (WAN).

The medical institution system 10 performs imaging of an examination target part of a subject and storing of the captured medical images based on an examination order from a doctor in a medical department using a known ordering system. In addition, the medical institution system 10 allows a radiologist to interpret a medical image and create an interpretation report, and allows a doctor of a medical department that is a request source to view the interpretation report.

The medical institution system 10 includes an imaging apparatus 11, an interpretation workstation (WS) 12 which is an interpretation terminal, a medical care WS 13, an image server 14, and a report server 16. Further, the medical institution system 10 may include a known information system such as a radiology information system (RIS) 18 and a hospital information system (HIS) 19. The imaging apparatus 11, the interpretation WS 12, the medical care WS 13, the image server 14, the report server 16, the RIS 18, and the HIS 19 are connected to each other in a communicable state via a wired or wireless network 9.

Each apparatus is a computer on which an application program for causing each apparatus to function as a component of the medical institution system 10 is installed. The application program may be recorded on, for example, a recording medium, such as a digital versatile disc read-only memory (DVD-ROM) or a compact disc read-only memory (CD-ROM), and distributed, and be installed on the computer from the recording medium. In addition, the application program may be stored in, for example, a storage device of a server computer connected to the network 9 or in a network storage in a state in which it can be accessed from the outside, and be downloaded and installed on the computer in response to a request.

The imaging apparatus 11 is an apparatus (modality) that generates a medical image showing a diagnosis target part of the subject by imaging the diagnosis target part. Examples of the imaging apparatus 11 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 11 is transmitted to the image server 14.

The interpretation WS 12 is a computer used by a medical worker, such as a radiologist in a radiology department, to interpret a medical image and create an interpretation report, and includes a processing device, a display device such as a display, and an input device such as a keyboard and a mouse. In the interpretation WS 12, a viewing request for a medical image to the image server 14, various types of image processing for the medical image received from the image server 14, display of the medical image, and input reception of a sentence regarding the medical image are performed. In the interpretation WS 12, analysis processing for medical images, support in creating an interpretation report based on the analysis result, a registration request and a viewing request for the interpretation report to the report server 16, and display of the interpretation report received from the report server 16 are performed. The above processes are performed by the interpretation WS 12 executing software programs for respective processes.

The medical care WS 13 is a computer used by, for example, a medical worker such as a doctor in a medical department to observe a medical image in detail, view an interpretation report, create an electronic medical record, and the like, and is configured to include a processing device, a display device such as a display, and input devices such as a keyboard and a mouse. In the medical care WS 13, a viewing request for the medical image to the image server 14, display of the medical image received from the image server 14, a viewing request for the interpretation report to the report server 16, and display of the interpretation report received from the report server 16 are performed. The above processes are performed by the medical care WS 13 executing software programs for respective processes.

The image server 14 is a general-purpose computer on which a software program that provides a function of a database management system (DBMS) is installed. The image server 14 is connected to an image database (DB) 15. In a case in which the image server 14 receives a request to register a medical image from the imaging apparatus 11, the image server 14 prepares the medical image in a format for a database and registers the medical image in the image DB 15. In addition, in a case in which the viewing request from the interpretation WS 12 and the medical care WS 13 is received, the image server 14 searches for a medical image registered in the image DB 15 and transmits the searched-for medical image to the interpretation WS 12 and to the medical care WS 13 that are viewing request sources.

The image DB 15 is realized by, for example, a storage medium such as a hard disk drive (HDD), a solid-state drive (SSD), and a flash memory. In the image DB 15, the medical image acquired by the imaging apparatus 11 and metadata related to the medical image are registered in association with each other. The connection form between the image server 14 and the image DB 15 is not particularly limited, and may be a form connected by a data bus, or a form connected to each other via a network such as a network-attached storage (NAS) and a storage area network (SAN).

The metadata may include, for example, identification information such as an image identification (ID) for identifying a medical image, a tomographic ID assigned to each tomographic image included in the medical image, a subject ID for identifying a subject, and an examination ID for identifying an examination. The metadata may also include information about the medical department that has requested the capture of the medical image. In addition, the metadata may include, for example, information related to imaging, such as a modality, an imaging method, an imaging condition, an imaging purpose, and an imaging date and time related to capturing a medical image. The “imaging method” and “imaging condition” are, for example, a type of the imaging apparatus 11, a manufacturing company, an imaging part, an imaging protocol, an imaging sequence, an imaging method, the presence or absence of use of a contrast medium, a slice thickness in tomographic imaging, and the like. In addition, the metadata may include information related to the subject such as the name, date of birth, age, and gender of the subject. The metadata may be acquired from, for example, the RIS 18 and the HIS 19.

The report server 16 is a general-purpose computer on which a software program that provides a function of a database management system is installed. The report server 16 is connected to the report DB 17. In a case in which the report server 16 receives a request to register the interpretation report from the interpretation WS 12, the report server 16 prepares the interpretation report in a format for a database and registers the interpretation report in the report DB 17. Further, in a case in which the report server 16 receives the viewing request for the interpretation report from the interpretation WS 12 and the medical care WS 13, the report server 16 searches for the interpretation report registered in the report DB 17, and transmits the searched-for interpretation report to the interpretation WS 12 and to the medical care WS 13 that are viewing request sources.

The report DB 17 is realized by, for example, a storage medium such as an HDD, an SSD, and a flash memory. In the report DB 17, an interpretation report created in the interpretation WS 12 is registered. The connection form between the report server 16 and the report DB 17 is not particularly limited, and may be a form connected by a data bus or a form connected via a network such as a NAS and a SAN.

Each device/apparatus included in the medical institution system 10 may be disposed in the same facility (for example, a hospital) or may be disposed in different facilities. In addition, the number of devices/apparatuses included in the medical institution system 10 is not particularly limited, and each device/apparatus may be composed of a plurality of devices/apparatuses having the same function.

The information processing apparatus 20 analyzes the interpretation report obtained in the medical institution system 10. The information processing apparatus 20 will be described below.

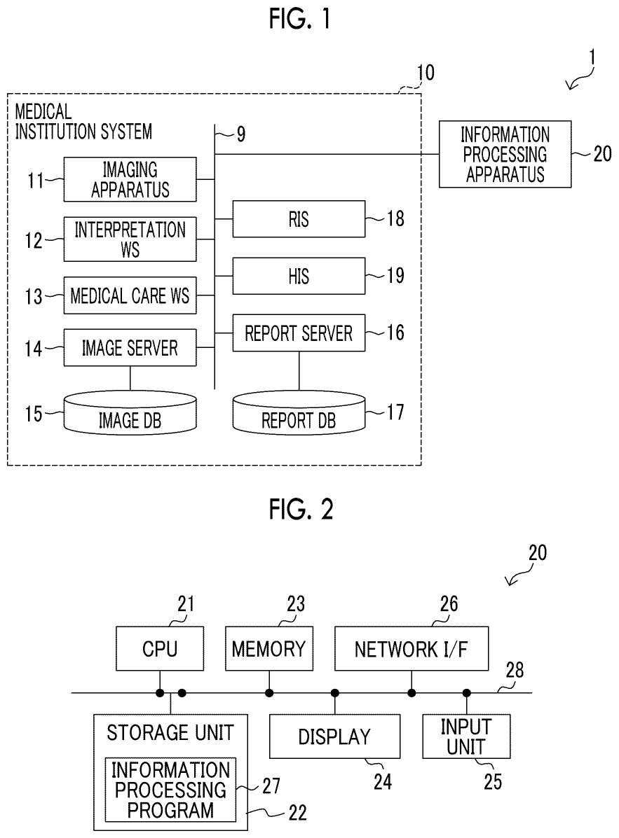

First, an example of a hardware configuration of the information processing apparatus 20 will be described with reference to FIG. 2. The information processing apparatus 20 includes a central processing unit (CPU) 21, a non-volatile storage unit 22, and a memory 23 as a temporary storage area. Further, the information processing apparatus 20 includes a display 24 such as a liquid-crystal display, an input unit 25 such as a keyboard and a mouse, and a network interface (I/F) 26. The network I/F 26 is connected to the network 9 and performs wired and/or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 20 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 20, for example, a personal computer, a server computer, a smartphone, a tablet terminal, a wearable terminal, or the like can be applied as appropriate.

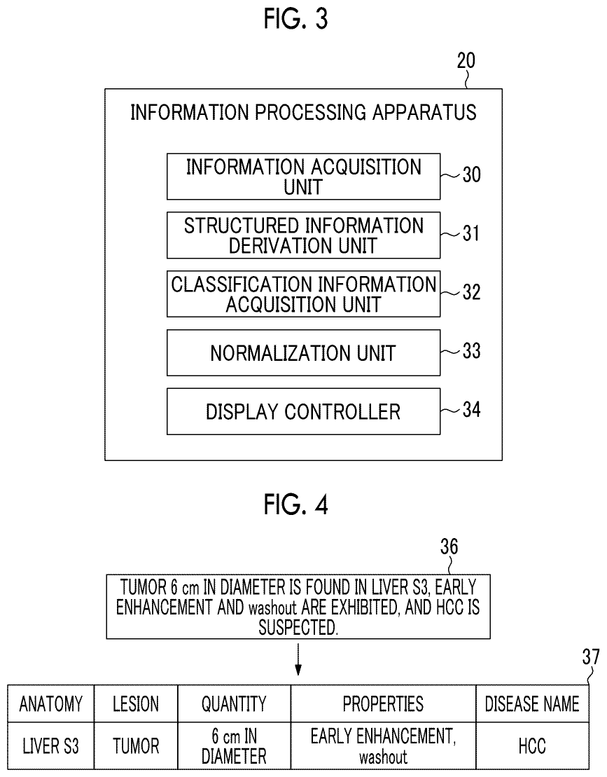

Next, an example of a functional configuration of the information processing apparatus 20 will be described with reference to FIG. 3. As shown in FIG. 3, the information processing apparatus 20 includes an information acquisition unit 30, a structured information derivation unit 31, a classification information acquisition unit 32, a normalization unit 33, and a display controller 34. The CPU 21 executes the information processing program 27, so that the CPU 21 functions as the information acquisition unit 30, the structured information derivation unit 31, the classification information acquisition unit 32, the normalization unit 33, and the display controller 34.

The information acquisition unit 30 acquires the interpretation report from an external device such as the report server 16. The interpretation report includes comments on findings in which the interpretation results are described. A comment on findings in which interpretation results included in the interpretation report are described is an example of a sentence in the present disclosure.

The structured information derivation unit 31 analyzes the interpretation report acquired by the information acquisition unit 30, extracts terms of each class, such as anatomy, lesion, quantity, properties, and disease name, from the comment on findings included in the interpretation report, to structure the comment on findings, and derives the terms of each class as structured information. As a method for extracting terms from a comment on findings, a known named entity extraction method using a natural language processing model such as, for example, bidirectional encoder representations from transformers (BERT) described in JP2023-114341A can be applied as appropriate.

For example, the structured information derivation unit 31 derives, from the comment on findings, the anatomy of a liver organ S1 and a sacral vertebra S2, and the like, diseases such as liver cysts, nodules, and ground glass opacities, quantities such as 1 cm and 5 mm, properties such as irregularity and calcification, and names of lesions such as fractures and lung cancer, as structured information.

Specifically, as shown in FIG. 4, in a case in which a comment on findings 36 included in the interpretation report is “A tumor 6 cm in diameter is found in a liver S3, early enhancement and washout are exhibited, and HCC is suspected”, the structured information derivation unit 31 derives, as structured information 37, “liver S3” as the anatomy, “tumor” as the lesion, “6 cm in diameter” as the quantity, “early enhancement and washout” as the properties, and “HCC (hepatocellular carcinoma)” as the disease name.

The classification information acquisition unit 32 acquires classification information regarding terms included in the interpretation report. The classification information includes at least one of synonyms of the structured information derived by the structured information derivation unit 31 or information on a field related to the interpretation report being analyzed. Information on a field related to the interpretation report being analyzed includes, for example, an organ including the lesion described in the interpretation report, a part of the organ, an imaging part (chest, head, and the like), a type of modality (CT apparatus, MRI apparatus, and the like), and a medical department (orthopedics, internal medicine, and the like) that has requested the capture of the images that created the interpretation report.

The interpretation report is associated with the medical image for which the interpretation report is created, and the medical image is associated with metadata. The metadata includes information such as the modality, the imaging part, and the medical department that has acquired the image for which the interpretation report is created, as described above. Alternatively, the interpretation report may include information such as the modality, the imaging part, and the medical department. Therefore, the classification information acquisition unit 32 can acquire classification information from the metadata of the medical image for which the interpretation report is created, or from this information included in the interpretation report. Further, the classification information acquisition unit 32 may be configured such that classification information is input by a user into the information processing apparatus 20 in advance and is stored in the storage unit 22, and the classification information acquisition unit 32 may acquire the stored classification information. The classification information acquisition unit 32 may also acquire classification information using a derivation model that has been subjected to machine learning to analyze the comment on findings and derive classification information (for example, organ names, part names, and the like).

For example, in a case in which an organ is acquired as classification information using a derivation model, from the comment on findings 36 shown in FIG. 4, “A tumor 6 cm in diameter is found in a liver S3, early enhancement and washout are exhibited, and HCC is suspected”, “liver organ” is acquired as classification information.

The normalization unit 33 normalizes the structured information based on the classification information acquired by the classification information acquisition unit 32, thereby deriving the representative notation for the structured information.

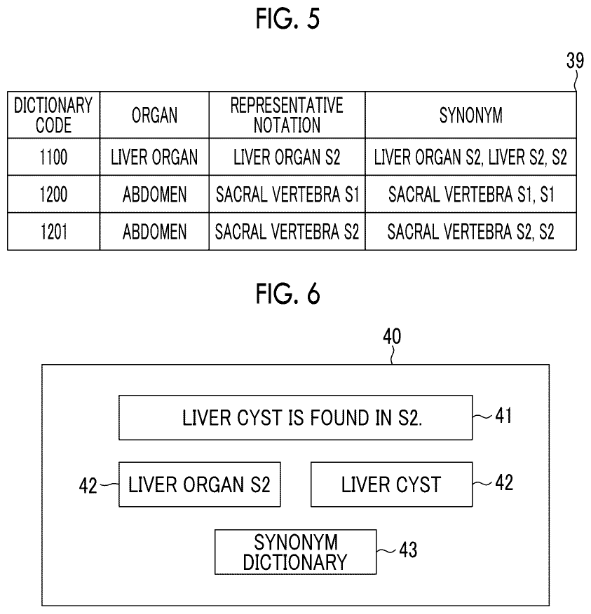

In the present embodiment, a synonym dictionary is used for normalization. FIG. 5 is a diagram showing an example of a synonym dictionary. As shown in FIG. 5, a synonym dictionary 39 associates a dictionary code, an organ, a representative notation, and a synonym with each other. In the synonym dictionary 39 shown in FIG. 5, in a dictionary code 1100, a liver organ, a liver organ S2, and “liver organ S2, liver S2, S2” are associated as an organ, a representative notation, and a synonym, respectively. In addition, in a dictionary code 1200, an abdomen, a sacral vertebra S1, and “sacral vertebra S1, S1” are associated as an organ, a representative notation, and a synonym, respectively. In addition, in a dictionary code 1201, an abdomen, a sacral vertebra S2, and “sacral vertebra S2, S2” are associated as an organ, a representative notation, and a synonym, respectively.

Here, it is assumed that the comment on findings is “A liver cyst is found in S2”. The structured information derivation unit 31 derives “S2” and “liver cyst” as the structured information. In a case in which the synonym dictionary 39 shown in FIG. 5 is referred to for the term “S2”, it is not possible to specify whether the representative notation is the liver organ S2 or the sacral vertebra S2.

In the present embodiment, the normalization unit 33 derives the representative notation based on the classification information. Here, in a case in which the classification information acquisition unit 32 analyzes the comment on findings “A liver cyst is found in S2” using a derivation model to acquire classification information, the derivation model outputs that the organ related to the comment on findings is the “liver organ” based on the description “liver cyst”. Therefore, the classification information acquisition unit 32 acquires “liver organ” as the classification information.

The normalization unit 33 refers to the synonym dictionary 39 shown in FIG. 5, and since the organ acquired as classification information for “S2” is the liver organ, acquires “liver organ S2” as the representative notation.

The display controller 34 displays the derivation result of the representative notations of the terms included in the interpretation report on the display 24. FIG. 6 is a diagram showing a display screen of the derivation result of the representative notation in the first embodiment. As shown in FIG. 6, a display screen 40 displays a comment on findings 41 and a derivation result 42 included in the interpretation report. The derivation result 42 includes “liver organ S2” and “liver cyst”. The display screen 40 displays text 43 of the “synonym dictionary” which is the basis used for normalization to derive the derivation result 42.

The derivation result is transmitted to the report server 16 and is stored in the report DB 17 in association with the interpretation report from which the derivation result is acquired.

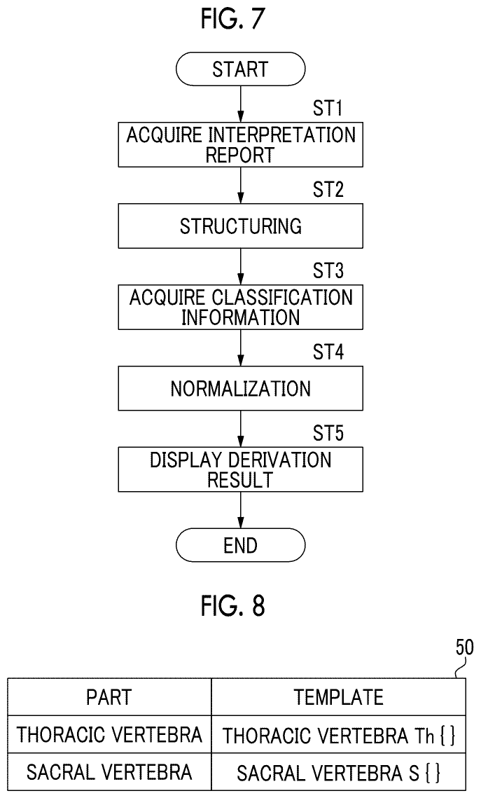

Next, processing performed in the first embodiment will be described. FIG. 7 is a flowchart showing the processing performed in the first embodiment. First, the information acquisition unit 30 acquires the interpretation report from the medical institution system 10 (Step ST1). Next, the structured information derivation unit 31 analyzes and structures the comment on findings included in the interpretation report to derive structured information (Step ST2).

Next, the classification information acquisition unit 32 acquires classification information regarding terms included in the interpretation report (Step ST3). Next, the normalization unit 33 normalizes the structured information based on the classification information to derive a representative notation (Step ST4). Then, the display controller 34 displays a display screen of the derivation result of the representative notation on the display 24 (Step ST5), and the process ends.

In this way, in the present embodiment, the representative notation of the structured term is derived by normalizing the structured information, which is the term included in the comment on findings, based on the classification information regarding the term included in the sentence. Therefore, even for a term for which a plurality of representative notations are derived in a case in which a synonym dictionary is referred to, one representative notation can be derived based on the classification information. Therefore, it is possible to perform normalization with high accuracy for terms extracted by structuring sentences.

Next, a second embodiment of the present disclosure will be described. Note that a configuration of an information processing apparatus in the second embodiment is the same as the configuration of the information processing apparatus in the first embodiment, only the processing to be performed is different, and thus the detailed description of the apparatus will be omitted. The second embodiment differs from the first embodiment in that in a case in which the normalization unit 33 normalizes the structured information, the normalization unit 33 refers to rules corresponding to the field related to the sentence.

Here, in a case in which the comment on findings included in the interpretation report is “Fractures are found in S1 and S2”, the structured information derivation unit 31 structures the comment on findings to acquire “S1”, “S2”, and “fracture” as structured information. In this case, the classification information acquisition unit 32 acquires “sacrum” as classification information from “fracture” and “S1” derived from the comment on findings. Further, the normalization unit 33 refers to the synonym dictionary 39 shown in FIG. 5 and derives a sacrum S1, whose synonym is “S1”, as the representative notation.

On the other hand, for the structured information “2”, even in a case in which the classification information is “sacrum”, the synonyms in the synonym dictionary do not include “2”. Therefore, the normalization unit 33 cannot normalize “2”, and as a result, it is not possible to derive a representative notation. In the second embodiment, before referring to a synonym dictionary, structured information is complemented with reference to rules corresponding to the field related to the sentence. In the second embodiment, it is assumed that a template is used as a rule corresponding to a field related to a sentence.

FIG. 8 is a diagram showing an example of a template used in the second embodiment. As shown in FIG. 8, a template 50 associates a part with a template. Specifically, “thoracic vertebra Th{ }” is associated with the thoracic vertebra as a template, and “sacral vertebra S{ }” is associated with the sacral vertebra as a template. Numbers are placed inside “{ }”. Th is an abbreviation for thoracic vertebra (thoracic), and S is an abbreviation for sacral vertebra (sacrum).

The normalization unit 33 specifies the part of the structured information “2” as the sacrum with reference to the classification information “sacrum” derived for “S1”. Next, the normalization unit 33 inserts a 2 into the “{ }” of the template of the sacral vertebra in the template 50, complements the “2” derived as the structured information, and acquires “sacral vertebra S2” as the complemented structured information. Then, “sacral vertebra S2” is derived as a representative notation for “sacral vertebra S2”, which is the structured information complemented with reference to the synonym dictionary.

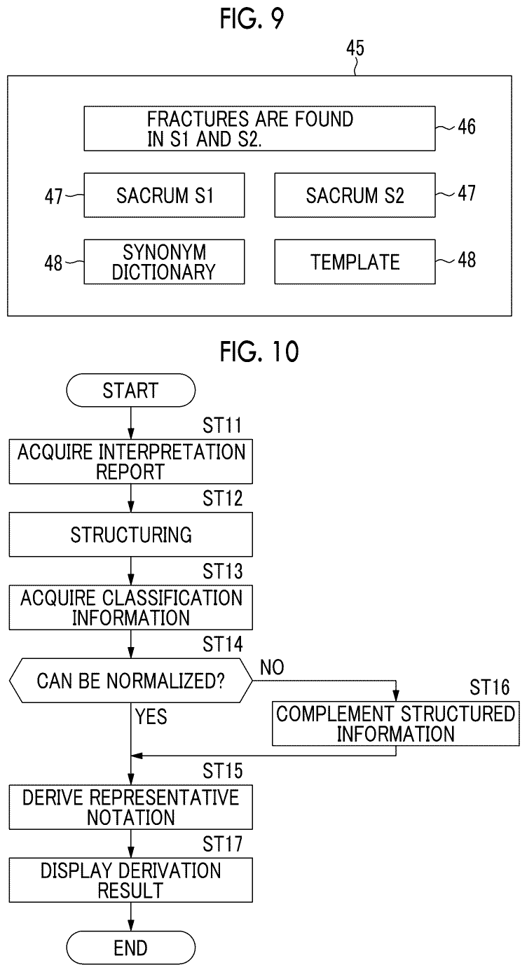

FIG. 9 is a diagram showing a display screen of the derivation result of the representative notation in the second embodiment. As shown in FIG. 9, a display screen 45 displays a comment on findings 46 and a derivation result 47 included in the interpretation report. The derivation result 47 includes “sacral vertebra S1” and “sacral vertebra S2”. The display screen 45 displays text 48 of the “synonym dictionary” and “template” which are the basis used for normalization to derive the derivation result 47.

Next, the second embodiment will be described. FIG. 10 is a flowchart showing the processing performed in the second embodiment. First, the information acquisition unit 30 acquires the interpretation report from the medical institution system 10 (Step ST11). Then, the structured information derivation unit 31 derives structured information by analyzing and structuring the comment on findings included in the interpretation report (Step ST12). Then, the classification information acquisition unit 32 acquires classification information regarding the terms included in the interpretation report (Step ST13). Next, the normalization unit 33 determines whether or not the structured information can be normalized with reference to the synonym dictionary based on the classification information (Step ST14).

In a case in which the result of Step ST14 is affirmative, the normalization unit 33 normalizes the structured information with reference to the synonym dictionary based on the classification information, thereby deriving a representative notation (Step ST15). In a case in which the result of Step ST14 is negative, the normalization unit 33 complements the structured information with reference to the template 50 (Step ST16), and proceeds to Step ST15 to normalize the complemented structured information, thereby deriving a representative notation. Then, the display controller 34 displays a display screen of the derivation result of the representative notation on the display 24 (Step ST17), and the process ends.

In this way, in the second embodiment, in a case in which structured information cannot be normalized with reference to a synonym dictionary based on classification information, the structured information is complemented with reference to a template. Therefore, by using the complemented structured information, it is possible to accurately normalize terms extracted by structuring a sentence.

In each of the above embodiments, there are cases in which structured information can be normalized without using classification information. For example, in a case in which the description of the comment on findings is “A liver cyst is found in liver organ S2”, the structured information is “liver organ S2” and “liver cyst”. In such a case, the classification information is “liver organ” for the organ, but even without using the classification information, with reference to a synonym dictionary, it is possible to derive “liver organ S2” as the representative notation. On the other hand, in a case in which the description of the comment on findings is “A liver cyst is found in S2”, as in the first embodiment, it is not possible to derive “liver organ S2” as the representative notation without using classification information.

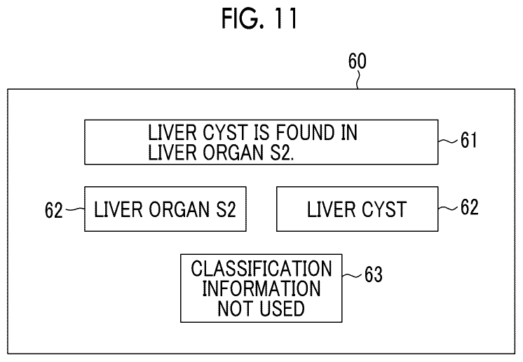

Therefore, the normalization unit 33 may determine whether or not structured information such as terms can be normalized without using classification information, and in a case in which the term can be derived, may normalize the structured information without using classification information. In this case, the display controller 34 may display, on the display screen of the derivation result, that the structured information has been normalized without using the classification information. For example, as shown in FIG. 11, on a display screen 60 of the derivation result, in addition to a comment on findings 61 of “A liver cyst is found in liver organ S2”, and a derivation result 62 (liver organ S2 and liver cyst), text 63 of “Classification information not used”, which indicates that classification information was not used, may be displayed. Displaying the text 63 is an example of displaying structured information normalized without using classification information and structured information normalized using classification information in a distinguishable manner, in the present disclosure.

In this case, the derivation result derived without using the classification information may be displayed in a different character color from the derivation result derived using the classification information, or the characters may be highlighted, and the like, to distinguish between the two.

Furthermore, in each of the above embodiments, a synonym dictionary, or a synonym dictionary and a template, is displayed as the basis for normalizing the structured information, but the present disclosure is not limited thereto. The basis for normalizing the structured information may not be displayed.

Furthermore, in the above embodiment, the target of structuring and normalizing is the comments on findings included in medical sentences such as interpretation reports, but the present disclosure is not limited thereto. The technology of the present disclosure can also be applied to structuring and normalizing any sentence other than medical sentences. For example, the technology of the present disclosure can be applied to a case in which a sentence including a place name is structured and normalized by converting the place name description included in the sentence into a search description (geographic coordinate value).

In the present embodiment, each process is executed by any computer. Further, any computer may execute these processes using a processor as hardware, a program as software, or a combination thereof. In such a case, the processor is configured to execute various types of processing according to the present embodiment in cooperation with the program, and can function as each unit or each means in the present embodiment. Further, the order in which the processes are executed by the processor is not limited to the order described above and may be changed as appropriate. Any computer may be a general purpose computer, a special purpose computer, a workstation, or other system capable of executing each process.

The processor may be configured with one or more pieces of hardware, and the type of hardware is not limited. For example, the processor may be configured using hardware, such as a central processing unit (CPU), a micro processing unit (MPU), a programmable logic device, such as a field-programmable gate array (FPGA), a dedicated circuit that is used to execute specific processing, such as an application-specific integrated circuit (ASIC), a graphic processing unit (GPU), or a neural processing unit (NPU). Further, the type of hardware may be a combination of different types of hardware. In a case in which a plurality of pieces of hardware are configured to execute one or more processes of a certain processor, the plurality of pieces of hardware may be present in devices physically separate from each other, or may be present in the same device. In addition, in any of the embodiments, the order of the processes performed by the processor is not limited to the order described above, and may be changed as appropriate. The hardware is configured using an electrical circuit (circuitry) in which circuit elements, such as semiconductor elements, are combined, or the like.

Further, the program may be software such as firmware or a microcode. The program may also be, for example, a group of program modules, each function of which may be implemented by a processor configured to execute each function. The program may be a program code or a plurality of code segments stored in one or more non-transitory computer-readable media (for example, storage media, other storages, or the like). The program may be stored in a plurality of non-transitory computer-readable media that are present in apparatuses physically separate from each other in a divided manner. The program code or the code segments may represent any combination of a procedure, a function, a subprogram, a routine, a subroutine, a module, a software package, a class, an instruction, a data structure, and a program statement. The program code or the code segments may be connected to other code segments or hardware circuits by transmitting and receiving information, data, an argument, a parameter, or content of a memory.

In the above embodiment, the information processing program 27 is described as being stored (installed) in the storage unit 22 in advance; however, the present disclosure is not limited thereto. The information processing program 27 may be provided in a form recorded on a recording medium, such as a compact disc read-only memory (CD-ROM), a digital versatile disc read-only memory (DVD-ROM), and a universal serial bus (USB) memory. Further, the information processing program 27 may be configured to be downloaded from an external device via a network.

The technology of the present disclosure extends to all kinds of program products. The program product includes all forms of products for providing a program. For example, the program product includes a program provided through a network such as the Internet, a non-transitory computer-readable recording medium, such as a CD-ROM, a DVD, and a USB memory in which the program is stored, and the like.

The supplementary notes of the present disclosure will be described below.

### Supplementary Note 1

An information processing apparatus comprising a processor,
in which the processor is configured to:
   acquire a sentence;
   derive structured information by structuring the sentence;
   acquire classification information regarding a term included in the sentence; and
   normalize the structured information based on the classification information.

### Supplementary Note 2

The information processing apparatus according to Supplementary Note 1,
in which the classification information includes at least one of a synonym of the structured information or information on a field related to the sentence.

### Supplementary Note 3

The information processing apparatus according to Supplementary Note 2,
in which the sentence is a medical sentence, and
the information on the field related to the sentence includes at least one of an organ, a part, a modality that has acquired a medical image for which the sentence is created, or a medical department that uses the medical image.

### Supplementary Note 4

The information processing apparatus according to Supplementary Note 1 or 2,
in which the processor is configured to normalize the structured information with reference to a synonym dictionary that has been classified according to the classification information.

### Supplementary Note 5

The information processing apparatus according to Supplementary Note 4,
in which the processor is configured to, in a case in which the structured information is not able to be normalized with reference to the synonym dictionary, complement the structured information using a rule corresponding to a field related to the sentence, and normalize the complemented structured information.

### Supplementary Note 6

The information processing apparatus according to any one of Supplementary Notes 1 to 5,
in which the processor is configured to output a basis for normalizing the structured information.

### Supplementary Note 7

The information processing apparatus according to any one of Supplementary Notes 1 to 6,
in which the processor is configured to display the normalized structured information.

### Supplementary Note 8

The information processing apparatus according to Supplementary Note 7,
in which the processor is configured to, in a case in which the structured information is able to be normalized without using the classification information, normalize the structured information without using the classification information, and display the structured information normalized without using the classification information and the structured information normalized using the classification information in a distinguishable manner.

### Supplementary Note 9

An information processing method comprising:
acquiring, by a computer, a sentence;
deriving, by the computer, structured information by structuring the sentence;
acquiring, by the computer, classification information regarding a term included in the sentence; and
normalizing, by the computer, the structured information based on the classification information.

### Supplementary Note 10

An information processing program causing a computer to execute:
a step of acquiring a sentence;
a step of deriving structured information by structuring the sentence;
a step of acquiring classification information regarding a term included in the sentence; and
a step of normalizing the structured information based on the classification information.

What is claimed is:

1. An information processing apparatus comprising a processor,
wherein the processor is configured to:
acquire a sentence;
derive structured information by structuring the sentence;
acquire classification information regarding a term included in the sentence; and
normalize the structured information based on the classification information.

2. The information processing apparatus according to claim 1,
wherein the classification information includes at least one of a synonym of the structured information or information on a field related to the sentence.

3. The information processing apparatus according to claim 2,
wherein the sentence is a medical sentence, and
the information on the field related to the sentence includes at least one of an organ, a part, a modality that has acquired a medical image for which the sentence is created, or a medical department that uses the medical image.

4. The information processing apparatus according to claim 1,
wherein the processor is configured to normalize the structured information with reference to a synonym dictionary that has been classified according to the classification information.

5. The information processing apparatus according to claim 4,
wherein the processor is configured to, in a case in which the structured information is not able to be normalized with reference to the synonym dictionary, complement the structured information using a rule corresponding to a field related to the sentence, and normalize the complemented structured information.

6. The information processing apparatus according to claim 1,
wherein the processor is configured to output a basis for normalizing the structured information.

7. The information processing apparatus according to claim 1,
wherein the processor is configured to display the normalized structured information.

8. The information processing apparatus according to claim 7,
wherein the processor is configured to, in a case in which the structured information is able to be normalized without using the classification information, normalize the structured information without using the classification information, and display the structured information normalized without using the classification information and the structured information normalized using the classification information in a distinguishable manner.

9. An information processing method comprising:
acquiring, by a computer, a sentence;
deriving, by the computer, structured information by structuring the sentence;
acquiring, by the computer, classification information regarding a term included in the sentence; and
normalizing, by the computer, the structured information based on the classification information.

10. A non-transitory computer-readable storage medium that stores an information processing program causing a computer to execute:
a step of acquiring a sentence;
a step of deriving structured information by structuring the sentence;
a step of acquiring classification information regarding a term included in the sentence; and
a step of normalizing the structured information based on the classification information.
